(19) United States
(12) Patent Application Publication
(10) Pub. No.: US 2023/0186469 A1
(43) Pub. Date: Jun. 15, 2023

(54) METHODS OF GRADING AND MONITORING OSTEOARTHRITIS

(71) Applicant: Alpha Intelligence Manifolds, Inc., New Taipei City (TW)

(72) Inventors: CHANG-CHOU YEN, New Taipei City (TW); WIN-HOW LEE, New Taipei City (TW); QING-ZONG TSENG, New Taipei City (TW); CHENG-WEI LIN, New Taipei City (TW)

(21) Appl. No.: 18/064,319

(22) Filed: Dec. 12, 2022

Related U.S. Application Data
(60) Provisional application No. 63/287,991, filed on Dec. 10, 2021.

(51) Int. Cl. G06T 7/00; A61B 5/00

(52) U.S. Cl. CPC G06T 7/0012; A61B 5/4528; A61B 5/4585; G06T 2207/30008

(57) ABSTRACT

The present invention relates to a method for improving the diagnostic accuracy of an artificial intelligence (AI) to diagnose osteoarthritis (OA). The method involves all or some steps of: generating a plurality of feature values from at least one input skeletal image, generating a quantitative Kellgren-Lawrence (KL) grade based on the plurality of feature values, and generating an explanation plot showing the contributions of each feature. The present invention also relates to a method of constructing a non-transitory computer-readable medium to perform the above tasks.

### RELATED APPLICATION

This application claims the benefit of the U.S. Provisional Application No. 63/287,991 filed on Dec. 10, 2021, titled “METHODS OF GRADING AND MONITORING OSTEOARTHRITIS,” which is incorporated herein by reference at its entirety.

### BACKGROUND OF THE INVENTION

### Field of the Invention

The present invention relates to a method for improving the diagnostic accuracy of an artificial intelligence (AI) to diagnose osteoarthritis (OA).

### Description of Related Art

Osteoarthritis (OA) is a degenerative joint disease, and is the most common type of arthritis. In this disease, inflammation and injury to the joint cause bony changes, deterioration of tendons and ligaments, and a breakdown of cartilage, resulting in pain, swelling, and deformity of the joint. Kellgren-Lawrence (KL) grading system is commonly used to classify the severity of OA. In this grading system, the affected joint is assigned a grade from 0 to 4, which is correlated with increasing severity of OA, with grade 0 signifying no presence of OA and grade 4 signifying severe OA. The table below provides the definition of each grade.

TABLE 1
The definition of KL grades

| KL grade | Definition |
|---|---|
| 0 | Definite absence of x-ray changes of osteoarthritis |
| 1 | Doubtful joint space narrowing and possible osteophytic |
| | lipping |
| 2 | Definite osteophytes and possible joint space narrowing |
| 3 | Moderate multiple osteophytes, definite narrowing of joint |
| | space and some sclerosis and possible deformity of bone |
| | ends |
| 4 | Large osteophytes, marked narrowing of joint space, severe |
| | sclerosis and definite deformity of bone ends |

A patient is diagnosed as having osteoarthritis if the KL grade is 2 or higher. The Kellgren-Lawrence grading system, however, has some obvious defects, which limits its further applications. First, at least parts of the scoring process depend on the rater's subjective judgment to the features in the radiograph. In other words, different raters may assign different scores to the same radiograph, and there is no objective standard to mitigate this inconsistency. In addition, the KL score is only semi-quantitative, while the disease progression of OA is a continuous process. Therefore, the KL grade only provides some hint or general evaluation, instead of precisely reflecting the status of OA progression.

Therefore, an improved OA grading system is desired for OA diagnosis.

### SUMMARY OF THE INVENTION

To resolve the problems, the present invention provides a method to improve the existing KL grading system. The new grading system introduces a fractional part for the grades while maintains consistency with traditional KL grading system, thus provides a better diagnostic efficacy to osteoarthritis.

The method disclosed herein comprises: receiving, by a grading module implemented in a computer system, a plurality of feature values; and generating, by the grading module, a quantitative Kellgren-Lawrence (KL) grade based on the plurality of feature values. The plurality of feature values is derived from at least one input skeletal image, the plurality of feature grades is generated based on a set of analysis logic, the quantitative KL grade has an integer part and a fractional part; and the quantitative KL grade is used to diagnose osteoarthritis.

In one embodiment, before receiving the plurality of feature values by the grading module, the method further comprises: receiving, by the computer system, the at least one input skeletal image; and generating, by the computer system, the plurality of feature values from the at least one input skeletal image. Before generating the plurality of feature values, the method may further comprise: extracting, by the computer system, at least one recognition area from the at least one input skeletal image; and determining, by the computer system, a recognition result by the at least one recognition area. The recognition result determines the set of analysis logic.

In one embodiment, the plurality of feature values comprises at least one joint space narrowing (JSN) feature value and at least one osteophyte (OST) feature value. The plurality of feature values may further comprise all or some of joint space width (JSW) feature value(s), joint space area (JSA) feature value(s), sclerosis (SCL) feature value(s), alignment feature value(s), attrition feature value(s), and cyst feature value(s).

In one embodiment, the quantitative KL grade is generated by a first machine learning model implemented in the grading module. The first machine learning model may be trained with multiple predetermined KL grades and corresponding multiple sets of training feature values, wherein each of the multiple predetermined KL grades is predetermined based on one of multiple training skeletal images; and each corresponding set of the training feature values is derived from the same one of the multiple training skeletal images. In one embodiment, before training with the multiple predetermined KL grades and the corresponding multiple sets of training feature values, the training step may further comprises generating the multiple sets of training feature values based on the multiple training skeletal images. The first machine learning model may be trained by a boosted regression tree algorithm, such as XGBRegressor algorithm.

In one embodiment, the method of this invention further comprises generating, by a feature importance estimation module implemented in the computer system, a plurality of feature importance indicators based on the plurality of feature values. The feature importance estimation module may be built based on the multiple sets of training feature values and the grading module. The built feature importance estimation module may be a SHAP estimation model, and the plurality of feature importance indicators may be a plurality of SHAP values.

Another aspect of the present invention is to provide a non-transitory computer-readable medium having stored thereon a set of instructions that are executable by a processor of a computer system to carry out a method of generating a quantitative KL grade. The method of generating a quantitative KL grade comprises: receiving, by the computer system, at least one input skeletal image; generating, by the computer system, a plurality of feature values based on the at least one input skeletal image; and generating, by the computer system, the quantitative KL grade from the plurality of feature values. The generated quantitative KL grade has an integer part and a fractional part.

In one embodiment of the non-transitory computer-readable medium, the plurality of feature values comprises at least one joint space narrowing (JSN) value and at least one osteophyte (OST) value. The plurality of feature values may further comprise all or some of joint space width (JSW) feature value(s), joint space area (JSA) feature value(s), sclerosis (SCL) feature value(s), alignment feature value(s), attrition feature value(s), and cyst feature value(s).

Other objectives, advantages and novel features of the invention will become more apparent from the following detailed description when taken in conjunction with the accompanying drawings.

### BRIEF DESCRIPTION OF THE DRAWINGS

FIG. 1 shows the flowchart of the present invention.

FIG. 2 shows the definition of quantitative measurements to knee joint cavity.

FIG. 3 shows the minimum JSWs and the widths of femur/tibia used in standardization.

FIG. 4 shows a knee joint image with points predicted by the landmark check model. The 4 ROIs (LT, MT, LF, and MF) used in diagnosis are also shown here.

FIG. 5 shows the identified areas for joint space area (JSA) calculations.

FIG. 6 shows an example of X-ray image with knee joint region identified.

FIG. 7 is the quantitative KL (QKL) grade and the explanation plot generated based on the features in Example 4. Each feature value is shown on the left. JSN_1 means the first JSN feature value, JSA_1 means the first JSA feature value, and so on. The features are sorted based on their individual contributions predicted by the SHAP estimator.

FIG. 8 is the multi-class confusion matrix comparing the AI predicted QKL value and the true KL label of 1,277 testing images.

FIG. 9 is the binary confusion matrix comparing the OA diagnosis results of 1,277 testing images performed by AI and by experts.

FIG. 10A shows the X-ray image of a patient; FIG. 10B shows the QKL grade, and the explanation plot derived from the X-ray image of FIG. 10A.

FIG. 11A shows the X-ray image of the same patient, but 4 years after the X-ray image in FIG. 10A was taken; FIG. 11B shows the QKL grade, and the explanation plot derived from the X-ray image of FIG. 11A.

### DETAILED DESCRIPTION OF PREFERRED EMBODIMENTS

The terminology used in the description presented below is intended to be interpreted in its broadest reasonable manner, even though it is used in conjunction with a detailed description of certain specific embodiments of the technology. Certain terms may even be emphasized below; however, any terminology intended to be interpreted in any restricted manner will be specifically defined as such in this Detailed Description section.

The embodiments introduced below can be implemented by programmable circuitry programmed or configured by software and/or firmware, or entirely by special-purpose circuitry, or in a combination of such forms. Such special-purpose circuitry (if any) can be in the form of, for example, one or more application-specific integrated circuits (ASICs), programmable logic devices (PLDs), field-programmable gate arrays (FPGAs), graphics processing units (GPUs), etc.

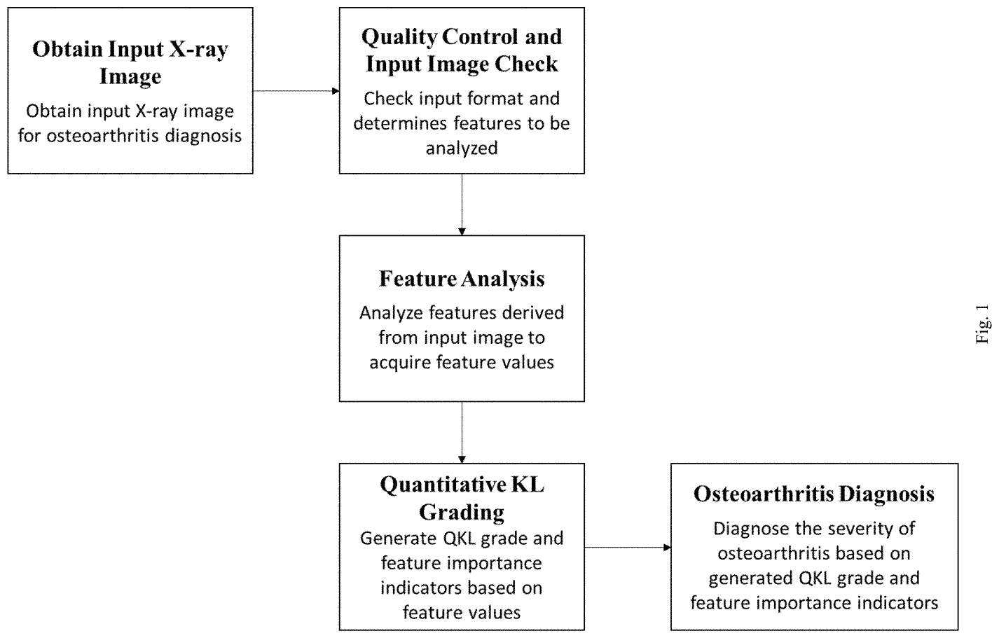

The flowchart of the present invention is provided in FIG. 1. To generate an OA grading system consistent with traditional KL grading, an artificial intelligence (AI) is trained to determine the KL grade of at least one input X-ray image. This AI is configured to identify and generate the feature values used in KL grading, such as joint space narrowing (JSN), osteophytes, sclerosis, and bone deformity. Other features which are not employed in traditional KL grading may also be included. The analyzed features may then be used to determine a quantitative OA grade with a fractional part. The feature identification and analysis process may be based on pre-defined rules, or may be established by machine learning algorithms. After the individual features are identified and analyzed, the generated feature values are integrated to generate a final OA grade. Below are the details of data processing and analysis.

### 1. Quality Control and Input Image Check

After receiving an input image, the first step is to check the image format, such as size, pixel value range, color format if applicable. In the case where a Digital Imaging and Communications in Medicine (DICOM) file is the input, the related DICOM tags, such as modality type and body part examined, may also be checked. In one embodiment, the DICOM tag of the input image is checked, and the analysis workflow is determined by the body part (e.g. knee, pelvis, spine) label in the DICOM tag. In an embodiment, the analysis is aborted if the DICOM tag is missing.

The next step is to use AI image analysis/recognition algorithms to check the quality of the input image. The AI algorithms may be a basic classification model trained by pre-labeled images to distinguish between good images and bad images, or it may be a classification model for specific purposes, such as distinguishing which part the image belongs to, estimating the shooting angle of the image, determining whether artificial joints and implants exist or not.

Besides classification models, a regression model may also be used to provide quality scores. One possibility is to label different images with numerical values (quality scores) during AI training. Another alternative method is to extract the results of network operations from a certain layer in the classification model as a quality score. For example, in the last layer of a multi-class classification model, a softmax function is usually used to normalize the probability of each category, making it sum to one, and then select the category with the highest value as the classification prediction result. This softmax score or its derived calculations can be used as the quality score. Alternatively, it is also possible to extract values from the layer output before calculating softmax normalization. These values are related to the degree of activation in the network for the image under specific categories, and can also be used to calculate the quality score.

An example of AI image analysis/recognition is to train a multi-class view classifier model, which is used to determine the part of body the image belongs to, the shooting angle of the image, and the orientation (left or right). In addition to outputting the classification results, it may also output the softmax value as the quality score.

In one embodiment, the AI image analysis/recognition comprises (1) a view check, (2) a region of interest (ROI) check and (3) a compatibility check. The view check is performed by a view classifier model to determine which part of body the input image belongs to. It may be executed by an AI trained by a collection of images labeled with their corresponding categories. The ROI check performs similarly but it is a little different from the view check, as the view check finds the category of the whole image, and the ROI check identifies the ROI block of a specific object (e.g. a knee joint or a hip) from the input image. The ROI check may be executed by another AI trained by a collection of images labeled with a specific object and the location of ROI within the images. The compatibility check is to determine whether the classification result from the view check and the ROI check coincide with each other. While it might be considered redundant to employ both view check and ROI check to classify the input image, the application of both checks increases the accuracy of overall QC, since the AI of the two checks learns differently. The probability that both view check and ROI check provide false result is small, thus the false prediction is minimized by checking whether the two checks predict the same result.

### 2. Feature Analysis

The image passed the quality control and determined suitable for OA diagnosis will be analyzed and graded by subsequent AI algorithms. An X-ray image showing the region of a joint may be determined suitable to proceed with the following feature analysis processes. For example, if the input image check identifies knee joint region(s) in the input image, the image may be proceeded with knee osteoarthritis (KOA) diagnosis. Alternatively, if the input image check identifies ankle joint region(s), the input image may be proceeded with ankle osteoarthritis diagnosis. Different kinds of osteoarthritis diagnosis utilize different features and indicators, such as analyzing different part of the body, and thus require different sets of analysis logic. “A set of analysis logic” described herein refers to a plurality of feature analyses used in a kind of osteoarthritis diagnosis.

Usually, an image passed the quality control and determined suitable for OA diagnosis is sufficient for grading by the AI algorithms. However, in some cases a plurality of X-ray images including the same body part may be used as the input. An example is two or more X-ray images of the same body part shot from different angles. One application of multiple input images is to construct a 3D-image or 3D parameters, and to generate a more accurate feature analysis result. Another possibility is to increase the sensitivity of the features related to OA progression, as described by Felson DT et al. (Approach Yields High Rates of Radiographic Progression in Knee Osteoarthritis. The Journal of Rheumatology 2008; 35: 2047-54.)

To be specific, for KOA diagnosis, the first model may be an object detection model to find the region of interest (bounding box) of the knee joint from the entire image. The second model may find the edge of the knee joint bone or specific anatomical locations from the region of interest (ROI) according to the results of the first model. The third model may use these points to calculate the joint space width (JSW). And the fourth model may use the points to calculate the femoral-tibial angle (FTA). The fifth model may label the areas of medial tibial, lateral tibial, medial femoral, lateral femoral and other local small areas derived from the points of previous models, and send them into models for classification or characteristic value prediction for OA grading. Examples include joint space narrowing (JSN), osteophyte, sclerosis, cyst, osteoporosis, bone mineral density (BMD) and so on. The details of the models will be given in the Examples below.

### 3. Quantitative Kellegren-Lawrence (KL) Grading

The results of the feature analyses, such as JSW, JSN, osteophyte, sclerosis and FTA alignment as described above, are integrated to generate a quantitative KL grade (QKL grade) by a trained AI model. This AI model may be a regression model trained by a set of X-ray images (training images) with KL grades labeled by experts. During training, the feature analysis results of the training images is the input, and the labeled KL grades is the ground truth corresponding to the input. To be consistent with the traditional KL grading, the generated QKL grade may be clipped to locate within the region of [0, 4]. In addition to QKL grade, a SHAP (SHapley Additive exPlanations) value may also be calculated to demonstrate the contribution of each feature. The QKL grading model and the SHAP estimator model will be described in detail in the Examples.

### EXAMPLES

The following examples are provided to further illustrate the details of generating feature values and the final OA grade for an input knee joint image.

### 1. Feature Analysis for Knee Osteoarthritis

In this embodiment, after the input image have passed the quality control and input image check identified knee joint region(s) in the input image, the following five factors are analyzed for knee osteoarthritis (KOA) diagnosis: (1) joint space widths (JSW), (2) joint space narrowing (JSN), (3) osteophyte (OST), (4) sclerosis (SCL), and (5) alignment. At least one feature value is generated for each factor. Some feature values are generated first, and used subsequently to generate additional feature values:

### (1) Joint Spaces Widths (JSW), Femur Width, and Tibia Width:

This part is quantitative measurements to knee joint cavity. The following parameters are calculated: JSW150, JSW175, JSW200, JSW225, JSW250, JSW275, JSW300, LJSW700, LJSW725, LJSW750, LJSW775, LJSW800, LJSW825, LJSW850, LJSW875, LSJW900, med mJSW value, lat mJSW value, femur condyle width, and tibia width. The definition of those parameters is described below.

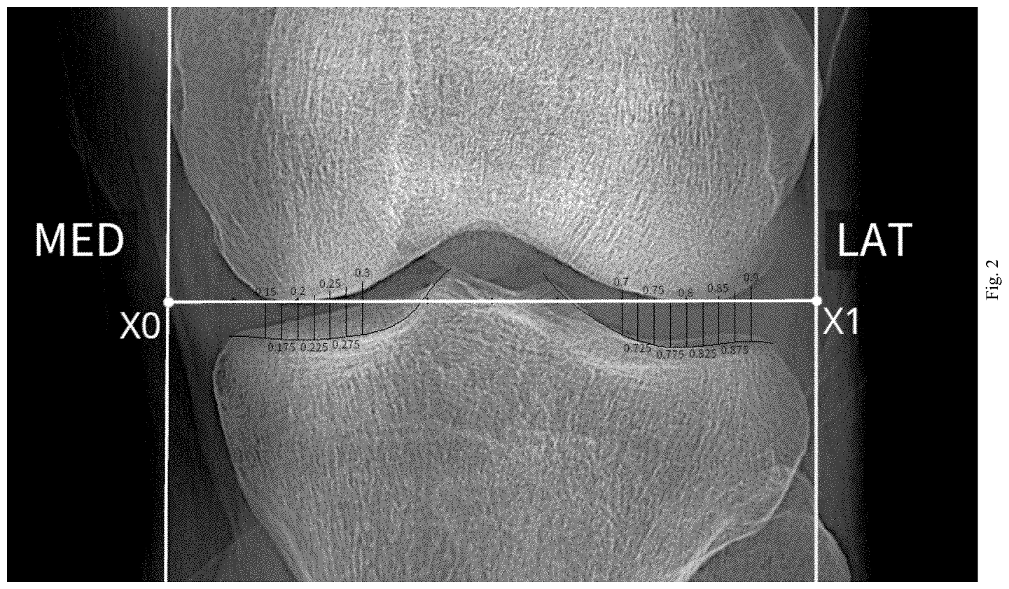

JSW150 to LJSW900 are the vertical distances between femur condyle and tibia plateau at positions of 0.15, 0.175, . . . 0.85, 0.875, and 0.90, respectively, as shown in FIG. 2.

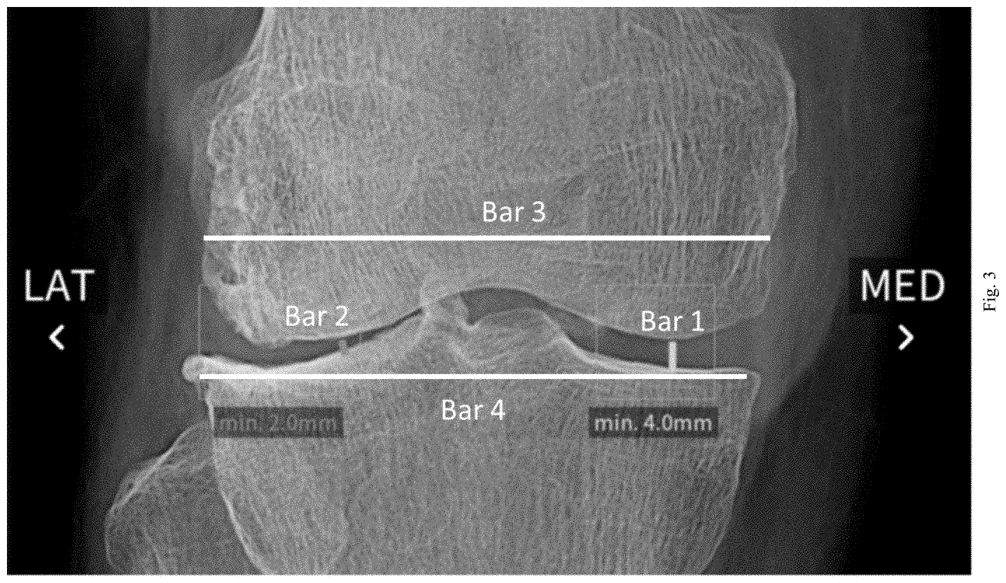

med mJSW (medial minimum JSW) is minimum measured distance between femur condyle and tibia plateau at the medial side, as bar 1 in FIG. 3 shows.

lat mJSW (lateral minimum JSW) is minimum measured distance between femur condyle and tibia plateau at the lateral side, as bar 2 in FIG. 3 shows.

Femur condyle width is the measured width of femur condyle, as bar 3 in FIG. 3 shows.

Tibia width is the measured width of tibia plateau, as bar 4 in FIG. 3 shows.

The calculated JSW can be standardized by the measured tibia width with the following formulae, as described by Paixao T et al. (A novel quantitative metric for joint space width: data from the Osteoarthritis Initiative (OAI). Osteoarthritis and Cartilage 2020; 28: 1055-61.)
the standardized joint Space Width:

$${JSW}_{a}^{std} = {JSW}_{a} \times \frac{\tilde{T}}{T}$$

T tibia width for this knee
T̅ is the average tibia width over OAI training data (generated via AIM)

The JSW can also be standardized by other reference values such as femoral condyle width, body weight, body height . . . etc.

A knee landmark model is used to identify the positions of knee joint. A trained a CNN model (HRnet) is used to predict those positions (landmarks). For each knee in the image, 74 keypoints are labeled. If those points are linked with a specified order, this set of points become a shape of the knee joint.

In training of knee landmark model, 1,500 labeled knees are used. Those 1,500 shapes (and thus all the keypoint sets) are aligned via rotation, scaling, shifting until the distance between each shape (each keypoint to the corresponding keypoints on other shapes) became minimum. The generated shape is called the average shape. In the domain of statistical shape model (SSM), this process is called Procrustes analysis. The 1,500 shapes after the Procrustes analysis provides a mean position and standard deviation (std) for each keypoint (i.e. each keypoint has its own mean position and standard deviation).

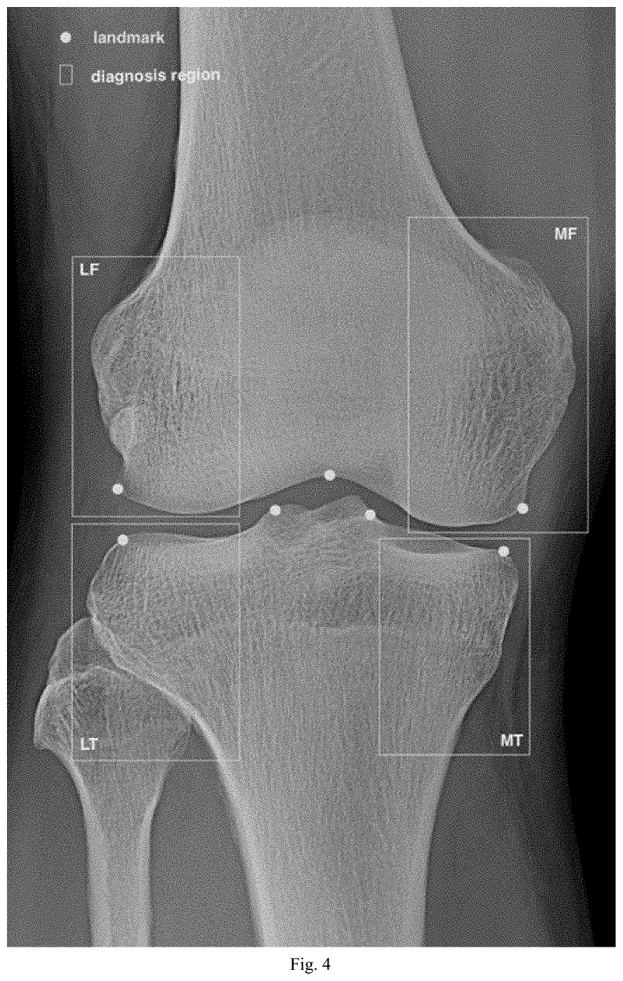

FIG. 4 shows a knee joint image with points predicted by the landmark check model. These points depict a contour, which is used to calculate JSW as described above.

### (2) Joint Space Narrowing (JSN):

This is to evaluate the severity of narrowing for the medial and lateral joint cavity. The input is the knee joint ROI inferred from the knee landmark predicted by the knee landmark model described above. Two feature values, medial JSN (med JSN) and lateral JSN (lat JSN), are generated by this model. To be consistent with the Osteoarthritis Research Society International (OARSI) grading criteria, the generated value lies between 0 and 4, where 0 is the least severe and 4 is the most severe.

The model performing this evaluation is a standard convolutional neural networks (CNN) model such as Resnet, EfficientNet, and Inception, to perform ordinal regression task. For model training, the JSN grades provided by the OAI dataset is used as the training dataset.

### (3) Osteophyte (OST):

This is to evaluate the severity of osteophytes at the positions of medial femur (MF), medial tibia (MT), lateral femur (LF), and lateral tibia (LT). To be consistent with the OARSI grading criteria, the output feature value is set between 0 to 4, where 0 is the least severe and 4 is the most severe.

Similar to JSN, the model performing this evaluation is a standard CNN model such as Resnet, EfficientNet, and Inception, to perform ordinal regression task. The inputs are 4 knee joint ROIs (LT, MT, LF, MF, as shown in FIG. 4) and the outputs are Osteophyte feature values for each ROI. This model is trained by the Osteophyte grades provided by the OAI dataset.

The OST parameters are further processed to generate the feature values of medial osteophyte (med OST) and lateral osteophyte (lat OST): med OST could be the average, maximum, minimum or any numerical processing using the original output: (OST-MF, OST-MT); and it is the same for lat OST.

### (4) Sclerosis (SCL):

This is to evaluate the severity of subchondral sclerosis at the positions of medial femur (MF), medial tibia (MT), lateral femur (LF), and lateral tibia (LT). To be consistent with the OARSI grading criteria, the output feature value is set between 0 to 4, where 0 is the least severe and 4 is the most severe.

Similarly, the model performing this evaluation is a standard CNN model such as Resnet, EfficientNet, and Inception, to perform ordinal regression task. The inputs are 4 knee joint ROIs (LT, MT, LF, MF, as shown in FIG. 4) and the outputs are Sclerosis feature values for each ROI. This model is trained by the Sclerosis grades provided by the OAI dataset.

The SCL parameters are further processed to generate the feature values of medial sclerosis (med SCL) and lateral sclerosis (lat SCL): med SCL could be the average, maximum, minimum or any numerical processing using the original output: (SCL-MF, SCL-MT); and it is the same for lat SCL.

### (5) Joint Space Area (JSA):

This is an additional feature value generated from JSW feature values by the following formulae:
JSA_{med}=Σᵢ₌₀⁵ JSA₁₅₀₊₂₅ᵢ¹⁷⁵⁺²⁵ⁱ
JSAₗₐₜ=Σᵢ₌₀⁵ JSA₇₀₀₊₁₅ᵢ⁷²⁵⁺²⁵ⁱ
JSAₐ^{b} is the Area between JSWa and JSWb
   for example, JSA₁₅₀¹⁷⁵ is the Area between JSW150 and JSW175

$${JSA}_{a}^{b} = \frac{1}{2} ( {JSW}_{a} + {JSW}_{b} ) \times {d}_{a , b} ( from Trapezoidal formula )$$

$${d}_{a , b} = \frac{b - a}{1000} \times femur condyle width$$

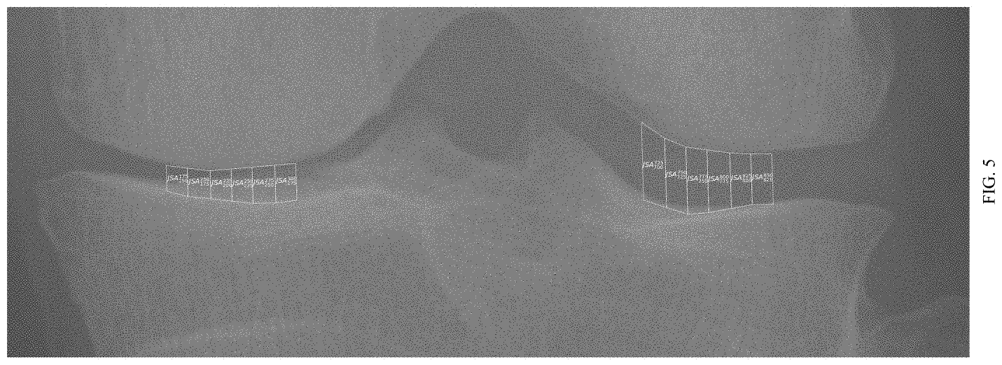

FIG. 5 shows the regions representing different JSA feature values. The JSA feature values can be further post-processed to generate other JSA related feature values, the post-processing could be taking the maximum, minimum, difference, and average of the JSA from the two compartments, as well as standardization process. The outputs from the JSA post-processing can be used as input feature values for quantitative KL generation.

### (6) Femoral-Tibial Angle (FTA) Alignment

It is the Femoral-Tibial angle measured using the predicted landmarks. It is not an AI/ML model (no training process involved). In the algorithms, basic computer vision and geometric calculation are performed based on the method described by: Iranpour-Boroujeni T, Li J, Lynch J, Nevitt M, Duryea J. A new method to measure anatomic knee alignment for large studies of OA: data from the Osteoarthritis Initiative. Osteoarthritis and Cartilage 2014 October; 22(10): 1668-1674.

### 2. Quantitative KL Grading

The feature values obtained from the above feature processing are used as the input for the generation of quantitative KL grade (QKL grade). This QKL grading model is a boosted regression tree algorithm (e.g. XGBRegressor). 6,000 to 40,000 knee joint X-ray images with expert-labeled KL grades are utilized during training. Each image is analyzed to obtain the predictions and measurements of the above-mentioned feature values, and therefore each X-ray has a predetermined (ground truth) KL grade and all the required feature values. The predetermined KL grades is the ground truth for the machine learning model, and the model is trained to predict a KL grade (quantitative KL grade) based on the associated input feature values.

Specifically, with all the above-mentioned features as the “XGBRegressor” model input (Xₙ) to predict the KL grade (y), the training process updates/optimizes the model parameters (such as number of trees, depth of trees, maximum number of leaves and so on) iteratively so that the difference between predicted QKL grade (y) and the true KL grade label (Y) becomes smaller. For loss calculation, mean square error (MSE) is used as the optimization target for training (fitting) this ensembled regression tree model. The loss function measures the average of (Y−y)² in each batch and tries to minimize it. After the model is trained to generate a QKL grade, a post processing is performed on the output prediction (clipping) to make it fall between 0 and 4. This is because there is no boundary condition set in the QKL model itself, where the training only tries to minimize the difference between y and Y.

The generated QKL grade has an integer part and a fractional part, which provides more accurate diagnosis of knee osteoarthritis compared to traditional KL grading (which provides only an integer grade).

3. AI Explanation Plot with SHAP Values

For a viewer to better understand what factor plays an important role in the predicted QKL grade, an AI explanation plot with SHAP values is subsequently generated by a SHAP estimator model after QKL grade generation.

The training input data (i.e. the input feature values) and the trained model for QKL grading are further used to build a SHAP estimator model. This SHAP estimator model is then able to calculate feature importance (i.e. the AI explanation plot with SHAP values) for each new QKL prediction instance by taking the new input feature values and the predicted QKL grade.

### 4. Real Example of Feature Analysis and QKL Grade Generation

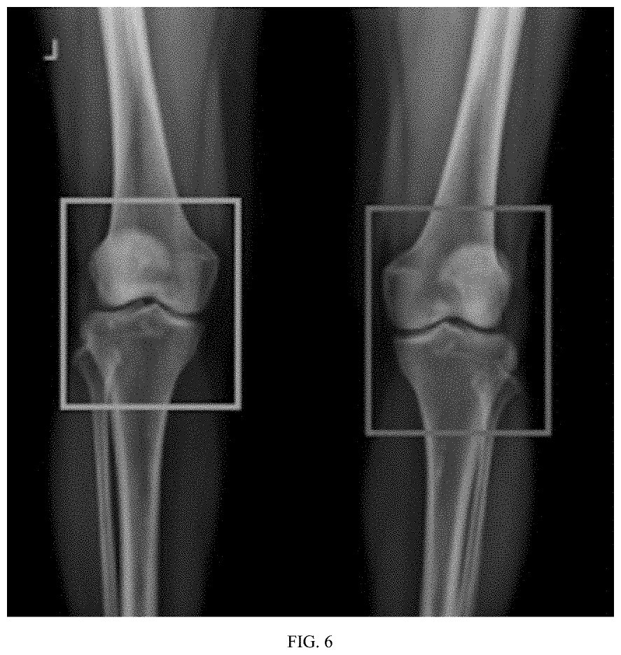

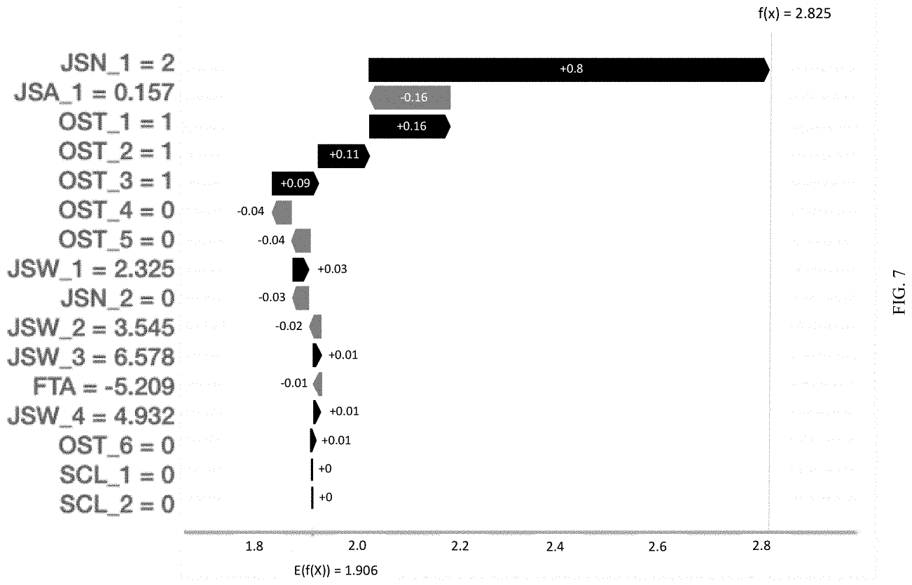

An X-ray image with knee joint region, as shown in FIG. 6, is used as the input for feature analyses. The output includes 4 JSW feature values generated by JSW model, 2 JSN feature values generated by JSN model, 6 OST feature values generated by OST model, 2 SCL feature values generated by SCL model, 1 JSA feature value generated by JSA model, and 1 FTA alignment feature value generated by FTA model. The above feature values are shown in FIG. 7 on the left.

The feature values generated above are used to predict a QKL grade and generate its corresponding AI explanation plot. The predicted QKL grade is 2.8, as shown in FIG. 7. The estimated contribution of each feature is also shown in FIG. 7. E[f(x)] is the mean estimation based on the input dataset used for building the SHAP model.

### 5. The Accuracy of QKL Grading Model

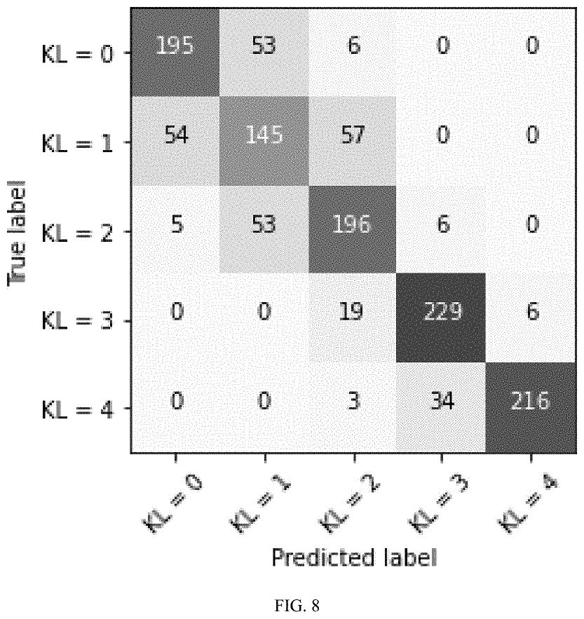

To evaluate the accuracy of the QKL grading model, 1,277 images from OAI dataset (which are not used during model training) are used as test set. The predicted QKL values rounded to nearest integers are compared to KL grades provided by experts. FIG. 8 is the resulting multi-class confusion matrix, which shows a good agreement between predicted QKL and expert-graded KL. The accuracy is 0.77 and the F1-score (macro average) is 0.77 for KL grading.

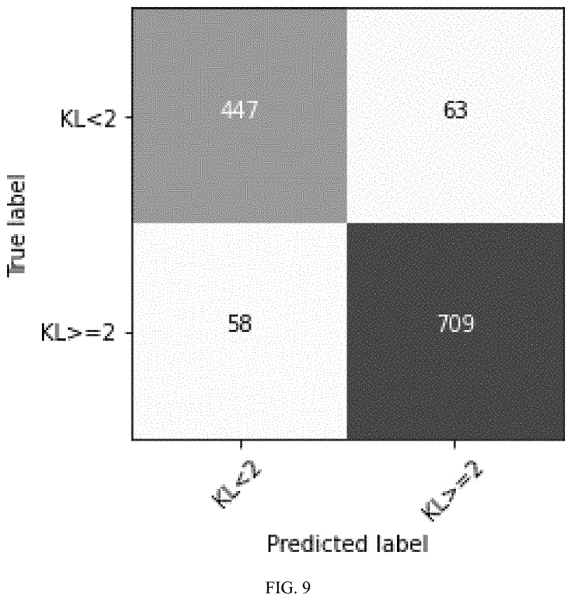

In clinical practice, a patient with KL grade >=2 is diagnosed as having definitive radiographic OA. The accuracy of QKL grading model for OA diagnosis is shown in FIG. 9, with the same 1,277 images as test data. The binary confusion matrix shows a sensitivity of 0.92 and a specificity of 0.88, indicating a good diagnosis efficacy.

### 6. The Diagnostic Power of QKL Grading Model

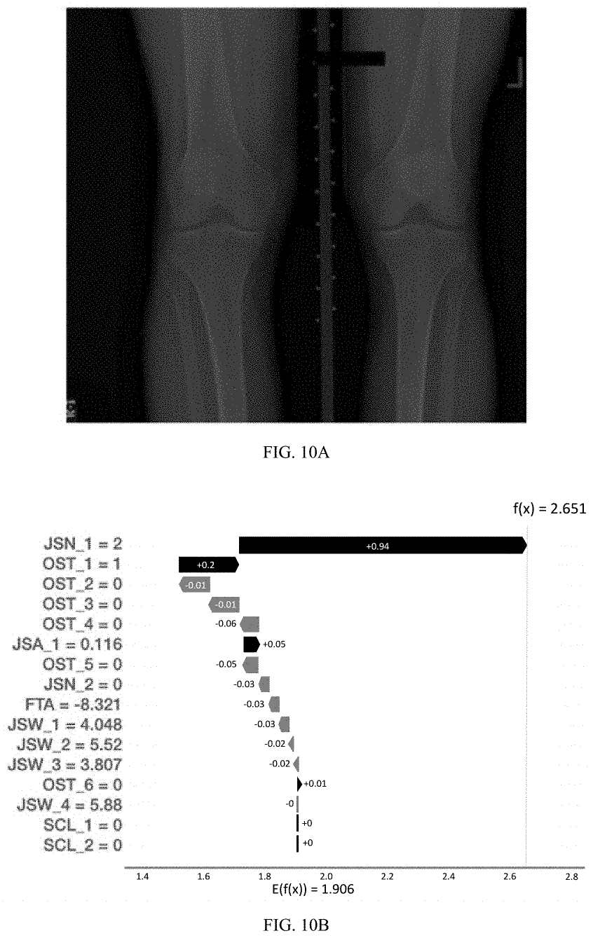

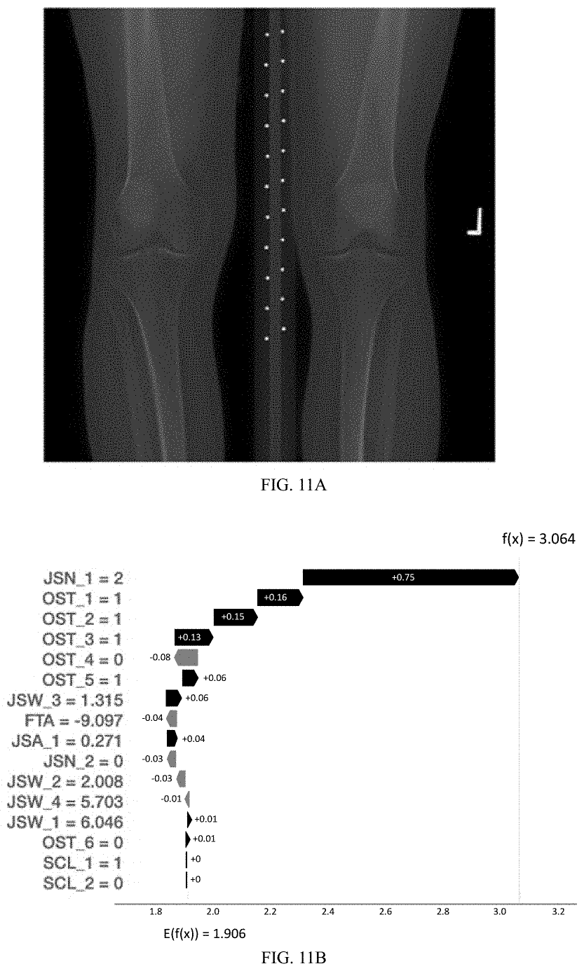

The superior diagnostic power of QKL grade compared to traditional grade can be shown in the following example. FIG. 10A and FIG. 11A are X-ray images of the same patient. The X-ray image in FIG. 11A is taken 4 years after that of FIG. 10A. The JSW_3 (with the value of 1.315) in FIG. 11B (with the value of 3.807 in FIG. 10B) means that the joint space is reduced, which can also be seen in the figures. However, according to the grading by experts and the definition of KL classification, both images are graded as KL3, and thus the disease progression cannot be observed by traditional KL grading. This difference can only be seen after introducing fractional KL grading. The QKL grade predicted by the model increased from 2.7 (FIG. 10B) to 3.1 (FIG. 11B).

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter set forth in the claims is not intended to be limited to the embodiments shown herein but is to be accorded the widest scope consistent with the principles and novel features disclosed herein. It is contemplated that additional embodiments are within the spirit and true scope of the disclosed subject matter. Thus, it is intended that the present invention covers modifications and variations that come within the scope of the appended claims and their equivalents.

What is claimed is:

1. A method for osteoarthritis diagnosis, comprising:
receiving, by a grading module implemented in a computer system, a plurality of feature values; and
generating, by the grading module, a quantitative Kellgren-Lawrence (KL) grade based on the plurality of feature values; wherein:
the plurality of feature values is derived from at least one input skeletal image;
the plurality of feature grades is generated based on a set of analysis logic;
the quantitative KL grade has an integer part and a fractional part; and
the quantitative KL grade is used to diagnose osteoarthritis.

2. The method of claim 1, before receiving the plurality of feature values by the grading module further comprising:
receiving, by the computer system, the at least one input skeletal image; and
generating, by the computer system, the plurality of feature values from the at least one input skeletal image.

3. The method of claim 2, before generating the plurality of feature values further comprising:
extracting, by the computer system, at least one recognition area from the at least one input skeletal image; and
determining, by the computer system, a recognition result by the at least one recognition area;
wherein the recognition result determines the set of analysis logic.

4. The method of claim 1, wherein the method is for knee osteoarthritis (KOA) diagnosis.

5. The method of claim 1, wherein the plurality of feature values comprises at least one joint space narrowing (JSN) feature value and at least one osteophyte (OST) feature value.

6. The method of claim 5, wherein the plurality of feature values further comprises at least one of:
one or more joint space width (JSW) feature values;
one or more joint space area (JSA) feature values;
one or more sclerosis (SCL) feature values;
one or more alignment feature values;
one or more attrition feature values; and
one or more cyst feature values.

7. The method of claim 1, wherein the quantitative KL grade is generated by a first machine learning model implemented in the grading module.

8. The method of claim 7, the training of the first machine learning model comprises training with multiple predetermined KL grades and corresponding multiple sets of training feature values, wherein:
each of the multiple predetermined KL grades is predetermined based on one of multiple training skeletal images; and
each corresponding set of the training feature values is derived from the same one of the multiple training skeletal images.

9. The method of claim 7, wherein the first machine learning model is trained by a boosted regression tree algorithm.

10. The method of claim 9, wherein the boosted regression tree algorithm is XGBRegressor algorithm.

11. The method of claim 8, before training with the multiple predetermined KL grades and the corresponding multiple sets of training feature values further comprising:
generating the multiple sets of training feature values based on the multiple training skeletal images.

12. The method of claim 7, wherein the first machine learning model is trained to output a predictive grade with an integer part and a fractional part.

13. The method of claim 8, further comprising:
generating, by a feature importance estimation module implemented in the computer system, a plurality of feature importance indicators based on the plurality of feature values.

14. The method of claim 13, wherein the feature importance estimation module is built based on the multiple sets of training feature values and the grading module.

15. The method of claim 13, wherein the feature importance estimation module is a SHAP estimation model, and the plurality of feature importance indicators is a plurality of SHAP values.

16. A non-transitory computer-readable medium having stored thereon a set of instructions that are executable by a processor of a computer system to carry out a method of generating a quantitative KL grade comprising:
receiving, by the computer system, at least one input skeletal image;
generating, by the computer system, a plurality of feature values based on the at least one input skeletal image; and
generating, by the computer system, the quantitative KL grade from the plurality of feature values;
wherein the quantitative KL grade has an integer part and a fractional part.

17. The non-transitory computer-readable medium of claim 16, wherein the plurality of feature values comprises at least one joint space narrowing (JSN) value and at least one osteophyte (OST) value.

18. The non-transitory computer-readable medium of claim 17, wherein the plurality of feature values further comprises at least one of:
one or more joint space width (JSW) feature values;
one or more joint space area (JSA) feature values;
one or more sclerosis (SCL) feature values;
one or more alignment feature values;
one or more attrition feature values; and
one or more cyst feature values.

19. The non-transitory computer-readable medium of claim 16, wherein the quantitative KL grade is generated by a first machine learning model implemented in the non-transitory computer-readable medium.

20. The non-transitory computer-readable medium of claim 19, wherein the first machine learning model comprises training with multiple predetermined KL grades and corresponding multiple sets of training feature values, wherein:
each of the multiple predetermined KL grades is predetermined based on one of multiple training skeletal images; and
each corresponding set of the training feature values is derived from the same one of the multiple training skeletal images.

21. The non-transitory computer-readable medium of claim 19, wherein the first machine learning model is trained by a boosted regression tree algorithm.
